SEQUENCE LISTING (12 sequences)

SEQ ID NO: 1 - DNA, 198 bp, Artificial
  Aritificial oligonucleotide
  misc_feature (31)..(38): n is a, c, g, or t
  misc_feature (100)..(107): n is a, c, g, or t
ccatctcatccctgcgtgtctccgactcagnnnnnnnnacactctttccctacacgacgctcttccgatctgatgcctatgacctgagatgttagatgannnnnnnnttcctggtgttacagcttcactaggagagatcggaagagcacacgtctgaactccagtcacctgagactgccaaggcacacaggggatagg

SEQ ID NO: 2 - DNA, 185 bp, Artificial
  Artificial oligonucleotide
  misc_feature (95)..(102): n is a, c, g, or t
caagcagaagacggcatacgagatcgagtaatgtgactggagttcagacgtgtgctcttccgatctgatgcctatgacctgagatgttagatgannnnnnnnttcctggtgttacagcttcactaggagagatcggaagagcacacgaacgacgactgagactgccaaggcacacaggggatagg

SEQ ID NO: 3 - DNA, 30 bp, Artificial
  454 reverse primer
cctatcccctgtgtgccttggcagtctcag

SEQ ID NO: 4 - DNA, 70 bp, Artificial
  Illumina forward primer
aatgatacggcgaccaccgagatctacactatagcctacactctttccctacacgacgctcttccgatct

SEQ ID NO: 5 - DNA, 56 bp, Artificial
  Illumina reverse primer
cctatcccctgtgtgccttggcagtctcagcagtcactcgtgtgctcttccgatct

SEQ ID NO: 6 - DNA, 56 bp, Artificial
  Illumina reverse primer
cctatcccctgtgtgccttggcagtctcagcaactgtgcgtgtgctcttccgatct

SEQ ID NO: 7 - DNA, 56 bp, Artificial
  Illumina reverse primer
cctatcccctgtgtgccttggcagtctcaggtcagatgcgtgtgctcttccgatct

SEQ ID NO: 8 - DNA, 56 bp, Artificial
  Illumina reverse primer
cctatcccctgtgtgccttggcagtctcagcttcatggcgtgtgctcttccgatct

SEQ ID NO: 9 - DNA, 56 bp, Artificial
  Illumina reverse primer
cctatcccctgtgtgccttggcagtctcagttggaaggcgtgtgctcttccgatct

SEQ ID NO: 10 - DNA, 56 bp, Artificial
  Illumina reverse primer
cctatcccctgtgtgccttggcagtctcagcacttgagcgtgtgctcttccgatct

SEQ ID NO: 11 - DNA, 56 bp, Artificial
  Illumina reverse primer
cctatcccctgtgtgccttggcagtctcaggaactgaccgtgtgctcttccgatct

SEQ ID NO: 12 - DNA, 56 bp, Artificial
  Illumina reverse primer
cctatcccctgtgtgccttggcagtctcagccttcgaacgtgtgctcttccgatct

(19) United States
(12) Patent Application Publication
(10) Pub. No.: US 2017/0253871 A1
(43) Pub. Date: Sep. 7, 2017

(54) METHOD OF PREPARING OLIGONUCLEOTIDE POOL USING ONE OLIGONUCLEOTIDE

(71) Applicant: UNIVERSITY-INDUSTRY FOUNDATION, YONSEI UNIVERSITY, Seoul (KR)

(72) Inventors: Du Hee BANG, Seoul (KR); Byung Jin HWANG, Seoul (KR)

(21) Appl. No.: 15/451,037

(22) Filed: Mar. 6, 2017

(30) Foreign Application Priority Data
Mar. 4, 2016 (KR) 10-2016-0026299

(51) Int. Cl. C12N 15/10; C12Q 1/68

(52) U.S. Cl. CPC C12N 15/1093; C12Q 1/6874

(57) ABSTRACT

Disclosed is a method of preparing an oligonucleotide pool having various combinations of base sequences using one oligonucleotide and next-generation sequencing (NGS). The oligonucleotide pool prepared according to the present invention does not require the use of conventional microarray chips which are expensive and time consuming, and improves efficiency in terms of cost and time by reducing the number of synthesis steps performed to obtain various combinations of oligonucleotides. In addition, according to the method of the present invention, the steps of preparing an oligonucleotide pool can be simplified, the number of combinations of oligonucleotides that can be prepared and utilized can be exponentially increased. Therefore, according to the method of the present invention, applications of genetic materials can be broadly extended.

### CROSS-REFERENCE TO RELATED APPLICATION

This application claims priority to and the benefit of Korean Patent Application No. 2016-0026299 filed on Apr. 3, 2016, the disclosure of which is incorporated herein by reference in its entirety.

### BACKGROUND

1. Field of the Invention

The present invention relates to a method of preparing an oligonucleotide pool having various combinations of base sequences, without the use of microarrays, using one oligonucleotide and next-generation sequencing (NGS).

2. Discussion of Related Art

As the amount of data in various fields rapidly increases, digital preservation is becoming more and more difficult. Most storage media, including magnetic and optical media, are easily degraded. In addition, as related businesses are growing rapidly, existing search and playback technologies are quickly becoming outdated.

Despite recent advances in the industry using programmable microarray synthesis capable of handling up to millions of oligonucleotides per chip, according to the current synthesis method using an array, to obtain a pool including a large number of oligonucleotides, a plurality of oligonucleotides having necessary sequences should be synthesized, which is expensive and labor-intensive. As a result, there is a problem in which a plurality of hypothesis tests should be performed using microarray synthesis. In addition, the fabrication of DNA microarray chips with a size of less than 1 micron (sub-micron) is very difficult, because parallel deposition of DNA bases must be precisely controlled to fabricate the microarray chips.

However, unlike the field of DNA writing, the cost of basic sequencing has fallen sharply over the past few years due to innovative technologies and competition between industries. Thus, it is inevitable that there is a widening gap in processing capacity between the field of DNA writing and the field of DNA reading, which may result in problems in expanding the use of synthetic DNA as a large-capacity genetic material or a memory material. Therefore, it is necessary to continuously study methods of synthesizing genetic materials more efficiently and economically.

### SUMMARY OF THE INVENTION

According to conventional methods of preparing a pool including a large number of oligonucleotides, synthesis using a microarray is required. Accordingly, since many kinds of oligonucleotides should be synthesized to fabricate a microarray chip, much time and effort are required. Therefore, the present invention has been made in view of the above problems, and it is an objective of the present invention to provide a novel method of preparing a pool of oligonucleotides having various combinations of base sequences without performing a step using a microarray chip. It is another objective of the present invention to provide a method of storing information in DNA using the method of preparing an oligonucleotide pool of the present invention, and a method of decoding the information.

In accordance with the present invention, the above and other objectives can be accomplished by the provision of a method of preparing an oligonucleotide pool including a plurality of clonal oligonucleotides, including a step of synthesizing a plurality of clonal oligonucleotides from one oligonucleotide; and a step of performing next-generation sequencing on the synthesized clonal oligonucleotides to identify the entire base sequence of each of the clonal oligonucleotides (wherein the step of synthesizing is performed so that the clonal oligonucleotide contains a random space, wherein the random space has a length of R mer and consists of any one base sequence selected from the group consisting of 4^{R} base sequences that can be made up of a combination of A, T, C, and G).

In accordance with an aspect of the present invention, the above and other objectives can be accomplished by the provision of a method of storing information in DNA, comprising a step of synthesizing a plurality of clonal oligonucleotides from one oligonucleotide; a step of performing next-generation sequencing on the synthesized clonal oligonucleotides to identify the entire base sequence of each of the clonal oligonucleotides; a step of performing mapping by inputting, on x-y coordinates, the base sequence of the random space of each of the clonal oligonucleotides, all base sequences of which have been identified; and a step of selecting, from a sequencing plate, a clonal oligonucleotide including a base sequence that matches a base sequence encoding information to be stored (wherein the step of synthesizing is performed so that the clonal oligonucleotide contains a random space, wherein the random space has a length of R mer and consists of any one base sequence selected from the group consisting of 4^{R} base sequences that can be made up of a combination of A, T, C, and G, and the base sequence of the random space consists of an address sequence encoding address information and a data sequence encoding data information).

In accordance with another aspect of the present invention, there is provided a method of decoding the information stored in DNA, including a step of encoding the information stored in DNA and decoding the same in the reverse direction.

In accordance with still another aspect of the present invention, there is provided an oligonucleotide consisting of a base sequence corresponding to SEQ ID NO: 1 or a base sequence corresponding to SEQ ID NO: 2.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a schematic diagram of the structure of an oligonucleotide for preparing an oligonucleotide pool, according to one embodiment of the present invention;

FIG. 2 shows a result of electrophoresis confirming products obtained by conducting annealing and extension on an oligonucleotide, according to one embodiment of the present invention;

FIG. 3 shows graphs representing the results of analyzing (A) read length distribution, (B) GC ratio, and (C) the number of times the same base sequence was read, after performing next-generation sequencing on a plurality of clonal oligonucleotides included in an oligonucleotide pool, according to one embodiment of the present invention;

FIG. 4 shows the result of a simulation that confirms coverage ratios for the combinations of the base sequences of clonal oligonucleotides that can be obtained depending on the number of NGS reads, according to one embodiment of the present invention;

FIG. 5 shows the result of a simulation that confirms the number of reads required to obtain the maximum contents by performing down-sampling on actual data, according to one embodiment of the present invention;

FIG. 6 shows the result of a simulation that confirms coverage ratios for the combinations of base sequences depending on the number of reads according to an increase in the base length of a random space, according to one embodiment of the present invention;

FIG. 7 is a schematic diagram showing a method of storing information in DNA according to one embodiment of the present invention;

FIG. 8 shows the result of a simulation that confirms that information to be stored increases as the base length of a random space increases, according to one embodiment of the present invention;

FIG. 9 shows a method of forming pools by duplicating encoded target information into eight tubes, according to one embodiment of the present invention; and

FIGS. 10a, 10b, 10c, 10d, 10e, 10f, 10g, and 10h show the results of analyzing the coverage of a pool formed in each of eight tubes, according to one embodiment of the present invention. The X-axis represents the coverage of each content with respect to encoding location, and the Y-axis represents the number of each coverage.

### DETAILED DESCRIPTION OF EXEMPLARY EMBODIMENTS

Unless otherwise defined, all terms (including technical and scientific terms) used herein have the same meaning as commonly understood by one of ordinary skill in the art. In general, the nomenclature used herein is well known and commonly used in the art.

In the present invention, the term “nucleotide” refers to DNA or RNA present in a single-stranded or double-stranded form, and the term “oligonucleotide” refers to a polymer in which the nucleotides are polymerized and thus may include polynucleotides and analogs thereof. In addition, in the present invention, the term “clonal oligonucleotide” refers to a product obtained through a synthesis process such as cloning or PCR using oligonucleotides. In the present invention, the product may be synthesized to include a random space using one oligonucleotide.

In the present invention, the term “amplification reaction” indicates a reaction for amplifying a target nucleic acid sequence, and may be performed using polymerase chain reaction (PCR). PCR includes reverse transcription polymerase chain reaction (RT-PCR), multiplex PCR, real-time PCR, assembly PCR, fusion PCR, and ligase chain reaction (LCR), without being limited thereto.

In the present invention, the term “primer” refers to one among single-stranded oligonucleotides, and may include a ribonucleotide, preferably a deoxyribonucleotide. The primer is hybridized or annealed at one site of a template to form a double-stranded structure. In the present invention, the primer may be hybridized or annealed to a NGS sequencing adaptor sequence. Annealing indicates that oligonucleotides or nucleic acids are juxtaposed to nucleic acid templates, and the juxtaposition allows polymerases to polymerize nucleotides to form a nucleic acid molecule complementary to a nucleic acid template or a portion thereof. Hybridization indicates that two single-stranded nucleic acids form a duplex structure by pairing complementary base sequences. The primer may act as a starting point of synthesis under conditions in which the synthesis of a primer extension product complementary to a template is induced.

In the present invention, the term “complementary” indicates that nucleic acids have enough complementarity to selectively hybridize to the nucleotide sequences under any particular hybridization or annealing condition.

In the present invention, the term “assembly of nucleotides” indicates that nucleic acid fragments with complementary base sequences are aligned and merged to form a longer nucleic acid fragment.

Hereinafter, the configuration of the present invention is described in detail.

In one aspect of the present invention, the present invention provides a method of preparing an oligonucleotide pool including a plurality of clonal oligonucleotides, including a step of synthesizing a plurality of clonal oligonucleotides from one oligonucleotide; and a step of performing next-generation sequencing on the synthesized clonal oligonucleotides to identify the entire base sequence of each of the clonal oligonucleotides.

According to the method of preparing an oligonucleotide pool of the present invention, a plurality of clonal oligonucleotides may be synthesized from one oligonucleotide, and the synthesis may be performed so that clonal oligonucleotides contain random spaces.

Conventionally, to synthesize a plurality of clonal oligonucleotides having various combinations of base sequences, a large number of oligonucleotides should be synthesized to prepare a microarray chip. However, this method requires a lot of cost and effort because a large number of oligonucleotides should be synthesized one by one. However, according to the present invention, it is possible to synthesize a plurality of clonal oligonucleotides having different base sequences from one oligonucleotide. Compared to conventional methods requiring at least several hundreds to tens of thousands of synthesis steps, the method of the present invention is advantageous in that oligonucleotide pools can be efficiently produced, and as the throughput of sequencing increases, the number of oligonucleotides that can be handled in fields using DNA molecules can be increased exponentially.

The synthesis may be performed so that the clonal oligonucleotide contains a random space. In addition, the step of synthesizing may be performed only once. Unlike the microarray method in which oligonucleotide sequences should be designed and synthesized as many times as necessary to obtain oligonucleotides having various combinations of base sequences, according to the method of the present invention, a single synthesis step is performed using an oligonucleotide having random spaces, and a plurality of clonal oligonucleotides having various combinations of base sequences are synthesized. Thus, synthesis steps required in the microarray method may be significantly reduced.

In the present invention, “random space” refers to a region consisting of any base sequence that can be generated by a combination of A, T, C, and G. Specifically, when a R mer-length random space is set in a starting oligonucleotide and synthesis is performed so that clonal oligonucleotides contain the random space, the base sequence of the random space may consist of one of base sequences that can be generated by all possible combinations of A, T, C, and G, and more specifically, may consist of any one base sequence selected from the group consisting of 4^{R} base sequences that can be made up of a combination of A, T, C, and G. Therefore, the plurality of clonal oligonucleotides may include two or more clonal oligonucleotides having different base sequences, and two or more clonal oligonucleotides having the same base sequence may be present.

The sequences of clonal oligonucleotides synthesized in the synthesis step are unknown. Specifically, a sequence except for a random space is the same as a starting oligonucleotide, but the base sequence of the random space is not known at the synthesis step. Thus, the method of the present invention includes a step of performing next-generation sequencing to identify the sequences of the synthesized clonal oligonucleotides.

In this respect, the R may be set differently depending on the length of oligonucleotides to be synthesized, the number of clonal oligonucleotides, the number of reads in next-generation sequencing, and the like, and the R is an integer and is not limited to a specific length. When a currently developed next-generation sequencer is used, R may cover up to 2 to 20, but is not limited to this range because of advancements in the throughput of next-generation sequencers.

In one embodiment of the present invention, in the case where the base sequence of a random space is 8 to 16 mer, the types of base sequences that can be obtained from up to 4^{R} combinations of base sequences were confirmed by performing experiments and simulations, and thus the types of clonal oligonucleotides containing the base sequences were confirmed. As a result, it was confirmed that as the number of reads increases, the number of combinations of base sequences to be covered increases (FIG. 6).

A method of synthesizing clonal oligonucleotides to contain the random spaces may be performed using a synthesis method conventionally used in the technical field of the present invention.

The “a plurality of clonal oligonucleotides” refers to two or more clonal oligonucleotides, and the clonal oligonucleotides synthesized in the synthesis step are preferably a population consisting of clonal oligonucleotides consisting of base sequences selected from all base sequences that can be generated by all possible combinations of A, T, C, and G. The population consisting of clonal oligonucleotides may include two or more clonal oligonucleotides having the same base sequence in a random space.

The “one oligonucleotide” refers to an oligonucleotide that is used to synthesize clonal oligonucleotides having various combinations of base sequences. In this respect, one oligonucleotide is used in combination with “a starting oligonucleotide”. The “one oligonucleotide” may be a group of oligonucleotides having the same base sequence, and may be designed and used differently depending on conditions, methods, apparatuses, and the like used for synthesizing clonal oligonucleotides and performing next-generation sequencing.

The “one oligonucleotide” may include one or more “N sequences (random space)”. The N sequences (random space) may be synthesized only with one of A, T, C, and G bases in the course of synthesizing clonal oligonucleotides. Thus, the N may be A, T, C or G in the oligonucleotide.

The “one oligonucleotide” may have a total length of 100 mer or more. When the length of the oligonucleotide of the present invention is 100 mer or more, sequencing may be facilitated because cluster formation occurs well in the step of identifying the sequences of clonal oligonucleotides using next-generation sequencing. The length may be less than 400 mer, without being limited thereto, and the optimum length may be determined according to the type of a next-generation sequencer, the throughput of a next-generation sequencer, and oligonucleotides consisting of desired combinations of base sequences.

To identify sequences using next-generation sequencing (NGS), both termini of the “one oligonucleotide” may include adaptor spaces in which adaptor sequences for next-generation sequencing are present. The oligonucleotide may include two or more adaptor sequences for use in more than one next-generation sequencer.

The adaptor sequence refers to a marker sequence that allows a next-generation sequencer to recognize base sequences, and the adaptor sequence applied to each device may differ depending on a method of using each device. For example, an adaptor sequence may be applied to the nucleotides of the present invention without limitation, as long as the adaptor sequence is applicable to next-generation sequencers including Roche's 454, Illumina's HiSeq, and Life Technologies (ABI)'s SOLiD. The adaptor sequence applicable to the next-generation sequencer may be sequences that are commonly used in the technical field of the present invention.

The “one oligonucleotide” may further include a dummy space consisting of a dummy sequence in addition to an adaptor space and a random space. The base sequence of the dummy space is preferably a homopolymer of 4 bp or less, and the ratio of G and C of the total bases in the dummy space may be 40 to 60%. When these conditions are satisfied, errors are reduced during the sequencing process and thus the efficiency of preparing oligonucleotide pools is increased.

In addition, the dummy space may include a random sequence of 1 to 10 mer in length. In this case, it is possible to prevent an optical crosstalk phenomenon from occurring when operating a next-generation sequencer, and thus the efficiency of the next-generation sequencer may be improved.

The “one oligonucleotide” may consist of a base sequence corresponding to SEQ ID NO: 1 or a base sequence corresponding to SEQ ID NO: 2.

In one embodiment of the present invention, an oligonucleotide of SEQ ID NO: 1 of 198 mer length was designed and clonal nucleotides were randomly synthesized and then sequencing was performed using Roche's 454 Junior system to identify the base sequence of each clonal oligonucleotide.

[SEQ ID NO: 1]
5′-
CCATCTCATCCCTGCGTGTCTCCGACTCAGNNNNNNNNACACTCTTTCCCT
ACACGACGCTCTTCCGATCTGATGCCTATGACCTGAGATGTTAGATGANNN
NNNNNTTCCTGGTGTTACAGCTTCACTAGGAGAGATCGGAAGAGCACACG
TCTGAACTCCAGTCACCTGAGACTGCCAAGGCACACAGGGGATAGG-3′

Specifically, an oligonucleotide, including each 454 adaptor sequence located in the 1st to 30th base positions and the 169th to 198th base positions from the 5′ terminus in SEQ ID NO: 1, a dummy space of 8 bp located in the 31st to 38th base positions to prevent the optical crosstalk phenomenon, a random space of 8 bp located in the 100th to 107th base positions, and other sequences such as a homopolymer of 4 bp or less and a dummy sequence having a GC ratio of 40 to 60%, was synthesized, and annealing was performed using a 30 bp reverse primer (SEQ ID NO: 4). The resulting double-stranded DNA was confirmed using electrophoresis.

In addition, in another embodiment of the present invention, an oligonucleotide of SEQ ID NO: 2 applicable to the Illumina platform was synthesized.

[SEQ ID NO: 2]
5′-
CAAGCAGAAGACGGCATACGAGATCGAGTAATGTGACTGGAGTTCAGAC
GTGTGCTCTTCCGATCT
GATGCCTATGACCTGAGATGTTAGATGANNNNNNNNTTCCTGGTGTTACA
GCTTCACTAGGAGAGATCGGAAGAGCACACGAACGACGACTGAGACTGC
CAAGGCACACAGGGGATAGG-3′

A step of identifying the entire base sequence of each of the clonal oligonucleotides of the present invention includes performing next-generation sequencing on a plurality of clonal oligonucleotides, which are randomly synthesized, and all base sequences of which are unknown.

When the clonal oligonucleotides including random sequences were synthesized using the oligonucleotide of the present invention, a plurality of clone oligonucleotides, all base sequences of which are unknown, may be obtained because the base sequences of the random sequence regions are unknown. Each of these clonal oligonucleotides, all base sequences of which are unknown, may be sequenced using next-generation sequencing, and thus an oligonucleotide pool may be generated.

The base sequences of random spaces present in the clonal oligonucleotides, all base sequences of which are unknown, are determined using next-generation sequencing. Conventionally, a method of synthesizing a plurality of clonal oligonucleotides by amplifying a plurality of oligonucleotides with known sequences has been used to prepare a pool. On the other hand, according to the present invention, an oligonucleotide pool having various combinations of base sequences may be prepared in a single synthesis process using an oligonucleotide, without the need to synthesize a plurality of oligonucleotides multiple times. Thus, the method of the present invention may increase economic and temporal efficiency and makes it easier to prepare an oligonucleotide pool.

Any kind of next-generation sequencer may be used as the next-generation sequencer of the present invention without limitation, and next-generation sequencing may also be performed by a method usually used in the field of the present invention.

In one embodiment of the present invention, clonal oligonucleotides were synthesized using an oligonucleotide of SEQ ID NO: 1 having a random space of 8 mer, and then the base sequences of the clonal oligonucleotides were identified using next-generation sequencing. Assuming that, when 100,000 reads out of a total of 4⁸ combinations of base sequences are obtained from sequencing, each clone is sequenced with a uniform probability, about 78% was obtained. In addition, it was confirmed by simulation that each clone may be obtained more uniformly when the number of reads is increased to 1 million and/or 10 million (FIG. 4).

The method of preparing an oligonucleotide pool according to the present invention may further include a step of selecting a clonal oligonucleotide including a random space consisting of a desired base sequence among a plurality of clonal oligonucleotides.

The “desired base sequence” refers to an intended base sequence. For example, when the desired base sequence is used as a base sequence or a storage medium that constitutes a part of a DNA molecule to be synthesized, the desired base sequence refers to a base sequence capable of encoding target information. The desired base sequence may be the base sequence of a random space included in a clonal oligonucleotide.

In the present invention, “selection” refers to selecting an oligonucleotide containing a desired base sequence among a plurality of oligonucleotides and may be used in combination with terms such as recovery and extraction.

The selection may be performed using a method of extracting or recovering oligonucleotides commonly used in the technical field of the present invention. For example, a selection system using a laser or a robot may be used, without being limited thereto.

In one embodiment of the present invention, nucleotides containing necessary data information are retrieved on a computer, and based on the positional information of the corresponding nucleotides, oligonucleotides having desired sequences or desired information are selected from oligonucleotides arranged in a form coupled with beads on a sequencing plate using a laser system.

Selection of bead-nucleotide conjugates using the laser system may be performed with reference to a method described in the following article: Howon Lee et al, A high-throughput optomechanical retrieval method for sequence-verified clonal DNA from the NGS platform, Nature Communications 6, Article number: 6073 (2015).

In another aspect of the present invention, the present invention provides a method of storing information in DNA, including a step of synthesizing a plurality of clonal oligonucleotides from one oligonucleotide; a step of performing next-generation sequencing on the synthesized clonal oligonucleotides to identify the entire base sequence of each of the clonal oligonucleotides; a step of performing mapping by inputting, on x-y coordinates, the base sequence of the random space of each of the clonal oligonucleotides, all base sequences of which have been identified; and a step of selecting, from a sequencing plate, a clonal oligonucleotide including a base sequence that matches a base sequence encoding information to be stored.

As described above, unless otherwise noted, the step of synthesizing a plurality of clonal oligonucleotides follows the above description.

The method of storing information in DNA may further include a step of encoding information (target information) to be stored in DNA as a base sequence made up of A, T, C, and G.

The target information includes all figures and texts in digital form, and is not limited by the format and type of information. Specifically, when codes constituting information can be converted into base codes, all of the corresponding information may be stored in DNA using the method of the present invention.

In one embodiment of the present invention, the target information was converted into Shannon information using the Huffman encoding method, and the target information was encoded as base sequences using a method of encoding each character as a DNA base sequence using base-4 digits (0, 1, 2 and 3 for T, C, G and A).

### Example Sentence

“The human genome holds an extraordinary trove of information about human development, physiology, medicine and evolution. Here we report the results of an international collaboration to produce and make freely available a draft sequence of the human genome. We also present an initial analysis of the data, describing some of the insights that can be gleaned from the sequence.”

The example sentence is 377 bytes of English text extracted from a key article in genomics and converted into Shannon information of 1569 bits (1). Next, the Shannon information was adjusted to the number of the base sequences of data information (2), and by applying the rules of Table 1, converted into bases matched with the numbers and encoded. For example, in the case of the first three words in the above text, when “The human genome” is transformed using a Huffman tree, the following result (1) is obtained.

“223313233233121032301020311” (1)

However, a location index and a data encoding part are 4 bp, respectively. Thus, when the result cannot be divided by 4, ‘0’ may be added at the end. The result of (2) is obtained by adding one ‘0’ in (1) and dividing the code into seven groups (length=28, 28/4=7).

{“2233”, “1323”, “3233”, “1210”, “3230”, “1020”, “3110” } (2)

Next, by adding a location code {‘0000’, ‘0001’, ‘0002’, ‘0003’, ‘0010’, ‘0011’, ‘0012’ } ahead of each code of (2), the following result of (3) is obtained.

“00002233”, “00011323”, “00023233”,

“00031210”, “00103230”, “00111020”, “00123110” (3)

TABLE 11

| | Target number | | | | |
|---|---|---|---|---|---|
| Types of | | 0 | 1 | 2 | 3 |
| bases | T | C | G | A | T |
| present in | C | G | A | T | C |
| front | G | A | T | C | G |
| | A | T | C | G | A |

The code of (3) is subject to the rules of Table 1, finally converted into a T, C, G or A base, and encoded into a DNA sequence. For example, when the first character starts with “T” (0) and the subsequent code is 2, the next nucleotide will be “A”.

The encoding method of the present invention is an example of a method of encoding target information into a base sequence, and the present invention may be performed using commonly used encoding methods and programs.

The “data information” refers to a value having meaningful information actually used in DNA synthesis or DNA information storage.

The “address information” refers to a value of encoding a location (order) in which the data information is present on the target information.

In the method of storing information in DNA, the base sequence of the random space may consist of an address sequence encoding address information and a data sequence encoding data information. The data information encoding the contents of target information has address information encoding the position (order) on the target information of the data information, which enables information to be read.

In the step of performing mapping by inputting, on x-y coordinates, the base sequence of the random space of each of the clonal oligonucleotides, all base sequences of which have been identified, the step may be performed by identifying the base sequence of the random space of each clonal oligonucleotide bound to a bead and inputting the position and data information of each of the clonal oligonucleotides on the x-y coordinates.

The “sequencing plate” is also referred to as “flow cell”, and refers to a plate to which oligonucleotides can bind. The surface of the sequencing plate may be in a lattice form, and thus may include a plurality of wells or cells. The well or cell may contain one or more beads capable of binding to oligonucleotides, and contains preferably one bead per well/cell.

According to the method of the present invention, when sequencing is performed, clonal oligonucleotides are each aligned on a sequencing plate in combination with one bead. By mapping the sequences of the random spaces of the aligned clonal oligonucleotides, it is possible to know where nucleotides having necessary data information are located on the sequencing plate. Thus, oligonucleotides having desired sequences may be rapidly selected from an oligonucleotide pool.

A method of aligning nucleotides on the sequencing plate and a method of converting the nucleotides into x-y coordinate values may be methods conventionally used in the field of the present invention.

The method of storing information in DNA includes a step of selecting clonal oligonucleotides. The step may be performed by selecting, from a sequencing plate, a clonal oligonucleotide including a data sequence that matches a desired base sequence.

The selection may be performed by method of searching for and recovering oligonucleotides commonly used in the technical field of the present invention. For example, a selection system using a laser or robot may be used, without being limited thereto.

In one embodiment of the present invention, nucleotides containing necessary data information are retrieved on a computer, and based on the positional information of the corresponding nucleotides, oligonucleotides having desired sequences or desired information are selected from oligonucleotides arranged in a form coupled with beads on a sequencing plate using a laser system.

A method of aligning nucleotides on the sequencing plate, a method of converting the nucleotides into x-y coordinate values, and a method of selecting bead-nucleotide conjugates using a laser system may be performed with reference to methods described in the following article: Howon Lee et al, A high-throughput optomechanical retrieval method for sequence-verified clonal DNA from the NGS platform, Nature Communications 6, Article number: 6073 (2015).

The “a data sequence that matches a desired base sequence” may refer to a base sequence identical or complementary to a base sequence necessary for constructing a DNA molecule to be synthesized. In addition, in the method of storing information in DNA or decoding information, a data sequence that matches a desired base sequence may be the same as or complementary to a sequence encoding information to be stored into information consisting of bases selected from the group consisting of A, T, C, and G.

The method of the present invention may further include a step of pooling selected clonal oligonucleotides in a DNA storage container.

The DNA storage container refers to a container capable of collecting DNA molecules, and it is preferable to use one capable of preventing the denaturation of and damage to DNA molecules, but the type of the container is not limited. For example, in one embodiment of the present invention, Falcon tubes were used as storage containers.

In the step of collecting DNA, nucleotides may be collected in one or more storage containers. To minimize errors in corruption of the data information by the bias of generated clonal oligonucleotides, a nucleotide pool capable of generating the same final products may be prepared in two or more DNA storage containers. In this case, each nucleotide sequence may include a barcode sequence so that each pool may be distinguished.

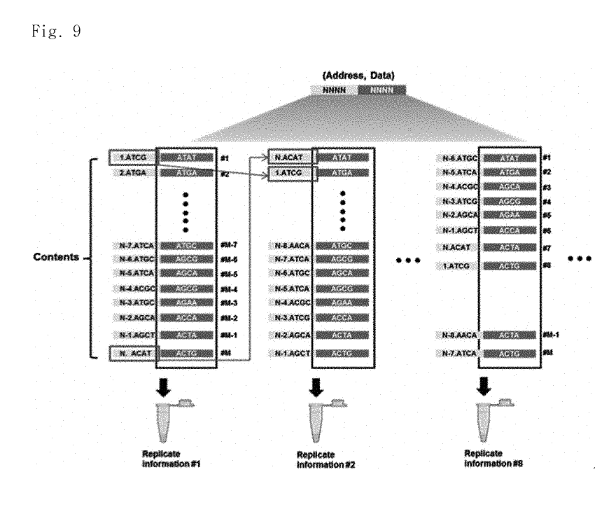

Specifically, referring to FIG. 9, there is a Falcon tube in which clonal nucleotides are tiled to have the same data sequence order. In tiling nucleotides, the same data sequences present in the same column have different address sequences. Therefore, when the base sequences of data sequences are identified by sequencing, in the case that there is an error in the data sequence of the nucleotide at the N_ACAT position, the data sequence at the N-1_AGCT position of the second tube may be read, which may reduce errors that may occur due to bias during sequencing. Such a method may be used in the method of storing information using clonal oligonucleotides prepared by the method of preparing an oligonucleotide pool according to the present invention, and one or more storage containers containing redundant information may be used to reduce damage or errors due to the bias of target information.

In addition, according to the present invention, the stored information may be restored by decoding the information stored in DNA in a reverse direction applying the same rules to the numbers corresponding to encoded bases. Accordingly, in another aspect, the present invention provides a method of decoding information, including a step of reading information stored in DNA.

Hereinafter, the present invention is described in detail with reference to preparation examples and experimental examples. The following examples and experiments are illustrative of the present invention and are not intended to limit the scope of the present invention.

### Examples

### [Preparation Example 1] Preparation of Single-Stranded Oligonucleotide

As shown in FIG. 1, an oligonucleotide was designed to include an 8 mer random N sequence and consist of a sequence totaling 198 bp, so that the clonal oligonucleotides of the present invention may include various combinations of base sequences.

SEQ ID NO: 1:
5′-
CCATCTCATCCCTGCGTGTCTCCGACTCAGNNNNNNNNACACTCTTTCCCT
ACACGACGCTCTTCCGATCTGATGCCTATGACCTGAGATGTTAGATGANNN
NNNNNTTCCTGGTGTTACAGCTTCACTAGGAGAGATCGGAAGAGCACACG
TCTGAACTCCAGTCACCTGAGACTGCCAAGGCACACAGGGGATAGG-3′

4 bp of the 8 bp (underlined part) N sequence of SEQ ID NO: 1 corresponds to an address portion (address sequence) in which the location of information is encrypted in the future, and the remaining 4 bp corresponds to a portion (data sequence) in which the content of actual information is encrypted. The 8 bp of the random N sequence at base positions 31st to 38th of SEQ ID NO: 1 were inserted to prevent the optical crosstalk phenomenon during sequencing with a Roche 454 Junior system. A dummy sequence was placed inside the Illumina sequencer adapter sequence, taking into account the case of the Illumina platform when the length of N increases afterwards. In addition, the dummy sequence was designed to have a homopolymer of 4 bp or less and a GC ratio of 40 to 60%. Sequencing adapter primer sequences of the Roche 454 Junior system applicable to current platforms were included in positions 1st to 30th and 169th to 198th.

Clonal oligonucleotides were synthesized randomly using an oligonucleotide consisting of the designed base sequence of SEQ ID NO: 1.

### [Example 1] Generation of Double-Stranded Oligonucleotides and Characterization Thereof

To prepare a library for sequencing, a mixture containing 1 μl of a 454 reverse primer (10 μM), 8 μl of distilled water, a reverse primer of SEQ ID NO: 3 for reducing PCR bias, and 10 μl of KAPA HiFi Hotstart ready mix (2×) was prepared, and then annealing and extension were performed on the mixture to generate double-stranded oligonucleotides.

Specific cycle conditions: Denaturation at 98° C. for 3 minutes; lowering the temperature to the annealing temperature (60° C.) at a rate of 0.1° C./sec; final extension at 72° C. for 5 minutes.

454 reverse primer
[SEQ ID NO: 3]
CCTATCCCCTGTGTGCCTTGGCAGTCTCAG

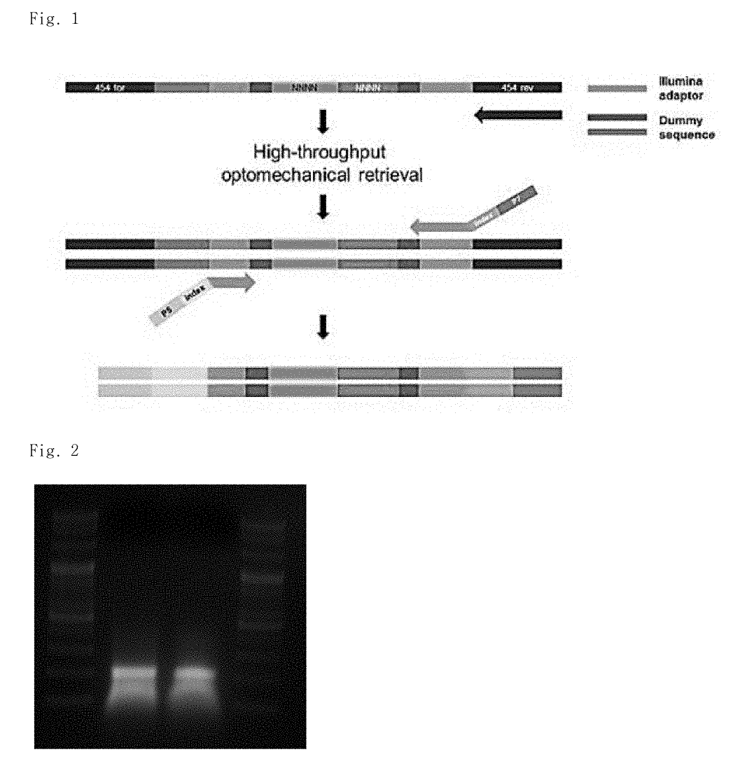

Then, the resulting DNA was electrophoresed on a 1.5% agarose gel to confirm size. The results are shown in FIG. 2.

### [Example 2] Analysis of Clonal Oligonucleotides

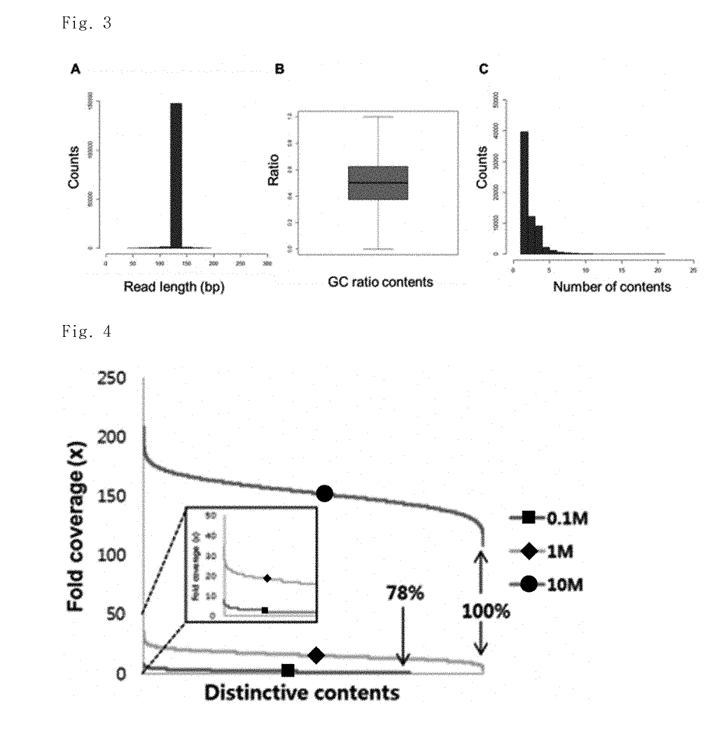

DNA obtained in Example 1 was applied to a Roche 454 Junior system (reading length˜400 bp) and sequenced to obtain clonal sequences (FIGS. 1 and 3). As a result, a total of 11,394,840 bp were obtained. After aligning to references using Bowtie2 version 2.2.4 (average alignment of 97.6% in end-to-end mode), then reading was filtered to an expected length (138 bp, 88.5% on average), and combinations of randomized 8-mer base sequences were further inspected for. Individual clonal sequences were extracted from a 454 sequencing plate using Sniper cloning, and oligonucleotides that were pooled in Falcon tubes were amplified using primers (eight combinations of forward and reverse NEBNext Mutiplex Oligos for Illumina, SEQ ID NO: 4 to SEQ ID NO: 12) containing specific index sequences for tiling information. Specifically, the primers used are shown in Table 2 below. As a forward primer, a primer of SEQ ID NO: 4 was used in the same manner, and as a reverse primer, a total of 8 different primer sets were used. Then, amplification was performed using the primer sets, and analysis was then performed.

TABLE 2

| SEQ ID NO: | | Base sequence |
|---|---|---|
| SEQ ID NO: 4 | Illumina forward | 5′- |
| | primer | AATGATACGGCGACCACCGAGATCTACACTATA |
| | | GCCTACACTCTTTCCCTACACGACGCTCTTCCG |
| | | ATCT-3′ |
| SEQ ID NO: 5 | Illumina reverse | CCT ATC CCC TGT GTG CCT TGG CAG TCT CAG |
| | primer | CAG TCA CTC GTG TGC TCT TCC GAT CT |
| SEQ ID NO: 6 | Illumina reverse | CCT ATC CCC TGT GTG CCT TGG CAG TCT CAG |
| | primer | CAA CTG TGC GTG TGC TCT TCC GAT CT |
| SEQ ID NO: 7 | Illumina reverse | CCT ATC CCC TGT GTG CCT TGG CAG TCT CAG |
| | primer | GTC AGA TGC GTG TGC TCT TCC GAT CT |
| SEQ ID NO: 8 | Illumina reverse | CCT ATC CCC TGT GTG CCT TGG CAG TCT CAG |
| | primer | CTT CAT GGC GTG TGC TCT TCC GAT CT |
| SEQ ID NO: 9 | Illumina reverse | CCT ATC CCC TGT GTG CCT TGG CAG TCT CAG |
| | primer | TTG GAA GGC GTG TGC TCT TCC GAT CT |
| SEQ ID NO: 10 | Illumina reverse | CCT ATC CCC TGT GTG CCT TGG CAG TCT CAG |
| | primer | CAC TTG AGC GTG TGC TCT TCC GAT CT |
| SEQ ID NO: 11 | Illumina reverse | CCT ATC CCC TGT GTG CCT TGG CAG TCT CAG |
| | primer | GAA CTG ACC GTG TGC TCT TCC GAT CT |
| SEQ ID NO: 12 | Illumina reverse | CCT ATC CCC TGT GTG CCT TGG CAG TCT CAG |
| | primer | CCT TCG AAC GTG TGC TCT TCC GAT CT |

As shown in FIG. 3, in the distribution in FIG. 3A, it was confirmed that the sequence of 138 bp excluding the 454 primer sequence (60 bp) at both ends of the oligonucleotide was mostly read well. Referring to FIG. 3B, the median value of a GC ratio was found to be distributed at 49%. When analyzing how well each clone was read (FIG. 3C), it was confirmed that most of the sequences were read less than 3 times (>80%), and the number of sequences read more than 20 times was extremely small.

### [Example 3] Analysis of Sequencing Results by Simulation

Since most of the clones were sequenced to three or fewer as shown in Example 1, the number of contents, that is, the number of clones, that could be obtained according to different amount of sequencing was confirmed by simulation.

As shown in FIG. 4, assuming each clone is sequenced with a uniform probability when 100,000 reads are obtained from sequencing, about 78% of the total maximum content 65,536 (=4⁸) was obtained. In addition, it was confirmed that each clone is obtained more uniformly when the number of reads increases to 1 million and/or 10 million.

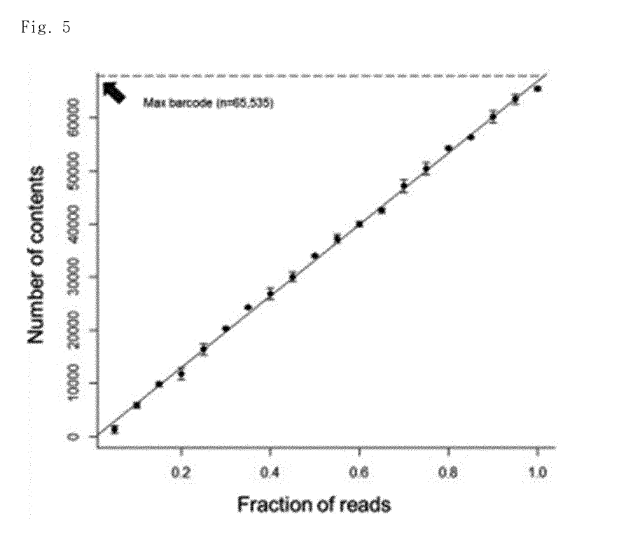

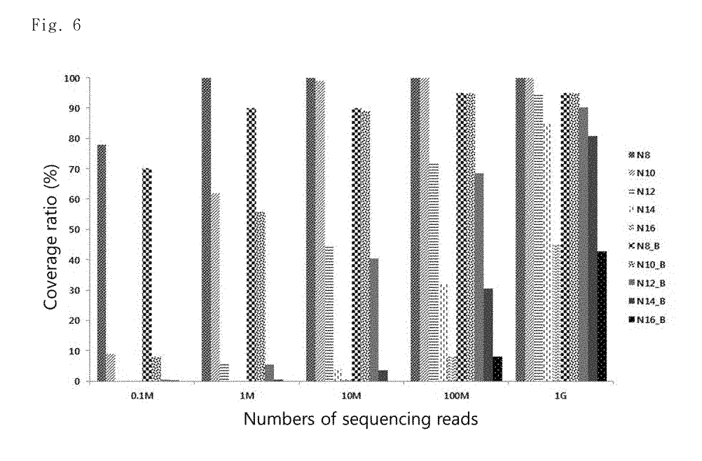

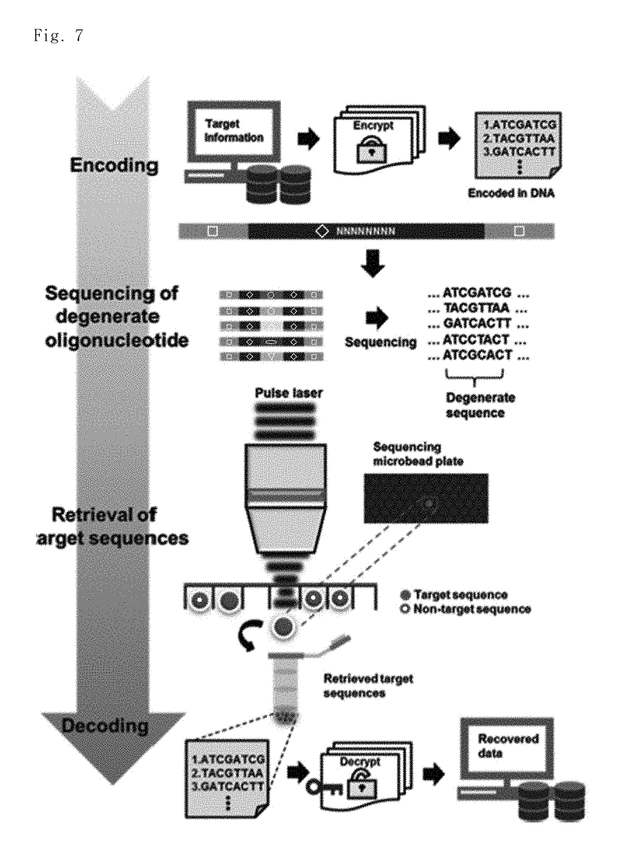

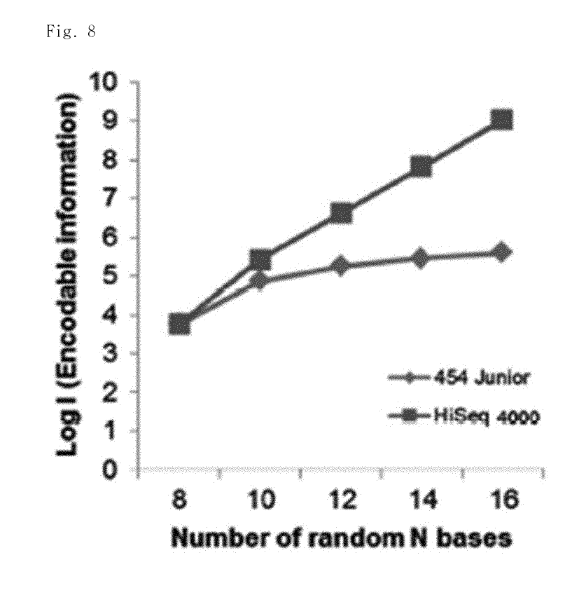

In addition, simulation was performed on necessary sequencing reads to obtain all the maximum possible content by down-sampling actual data. As shown in FIG. 5, as a result of repeating the down-sampling 10 times through Monte-Carlo simulation, it was confirmed that saturation state is reached even with the present sequencing result.

### [Example 4] Possibility of Platform Expansion

In Example 3, it was confirmed that most of the contents of the random N 8 bp oligonucleotides could be obtained in the case of the throughput of 454 sequencing (˜150,000 reads). Simulation experiments were conducted to determine the amount of contents that could be obtained when this length increased to 16 bp.

As shown in FIG. 6, as the space of random N capable of encoding increased, the ratio of the maximum obtainable contents also increased. In particular, in the case of the 8 bp space, the contents of all clones were obtained even though only one million reads were present. However, when each clone was not uniformly obtained and had a biased distribution, coverage of about 65% was observed with one million reads. In the case of an Illumina sequencer, the highest level of sequencing throughput currently available, output is close to 1G reads. In this case, it was confirmed by simulation that the combinations of base sequences that may be covered reaches 45% when N is extended to 16 bp.

### [Example 5] Verifying Applicability as DNA Memory Through Encoding and Reading

To confirm whether DNA may actually be used as a storage medium, the Huffman tree structure coding method was applied to the 377 characters (including letters, spaces, and special characters) described below, and Shannon information of 1569 bits were converted into Quaternary and encoded.

“The human genome holds an extraordinary trove of information about human development, physiology, medicine and evolution. Here we report the results of an international collaboration to produce and make freely available a draft sequence of the human genome. We also present an initial analysis of the data, describing some of the insights that can be gleaned from the sequence.”

The bit information of each file was encoded into a 4-bit data location block (4 nt) and a 4-bit data block (4 nt).

In addition, assuming that there were bases with synthesis or base sequence errors and that the synthesis process was stochastic, 8-fold coverage was designed by sliding an address block DNA sequence. To create redundant information, address bases are shifted to subsequent address block (FIG. 9). With this approach, the remainder of a sequence library may be utilized. Finally, these strings consisting of a quaternion (0, 1, 2 and 3) were converted to a single DNA base.

Next, an oligonucleotide of SEQ ID NO: 1 was annealed, sequenced, and then mapped and target sequences containing beads were recovered from a 454 sequencing plate using a high efficiency optomechanical system based on non-contact laser pulses. Specifically, the mapped 454 sequencing plate (chip) was placed in an optical retrieval system and beads-nucleotides were extracted using laser pulses. An amplification step was then performed and the initial mapping was evaluated with Sanger sequencing.

Then, a process of mapping the pixel information of sequences to the physical position of beads (x, y read locations registered in sequencing output) is performed. This allows the beads to be automatically separated using a laser system with the help of a linearized motor stage.

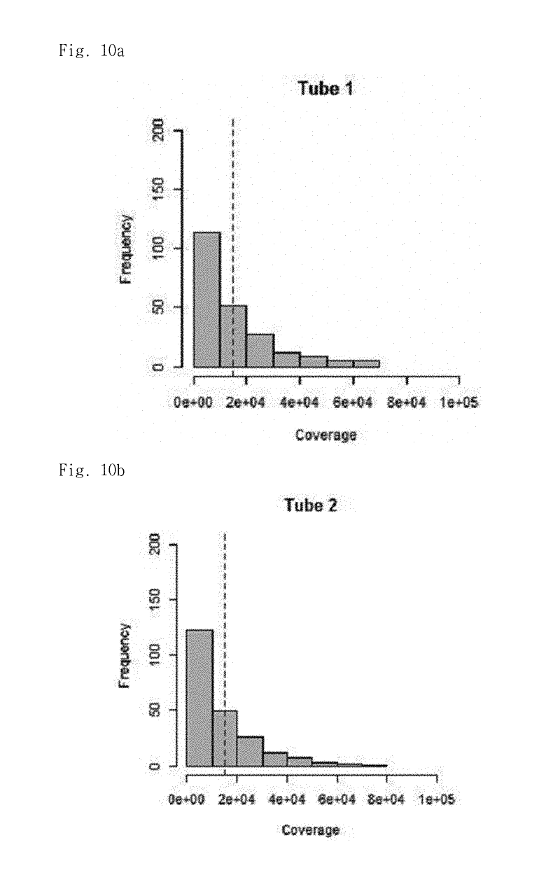

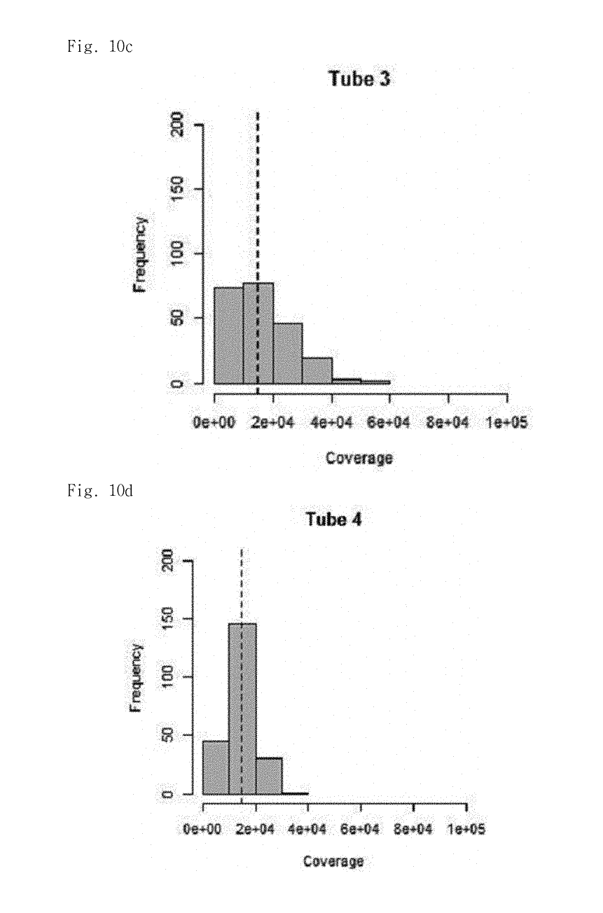

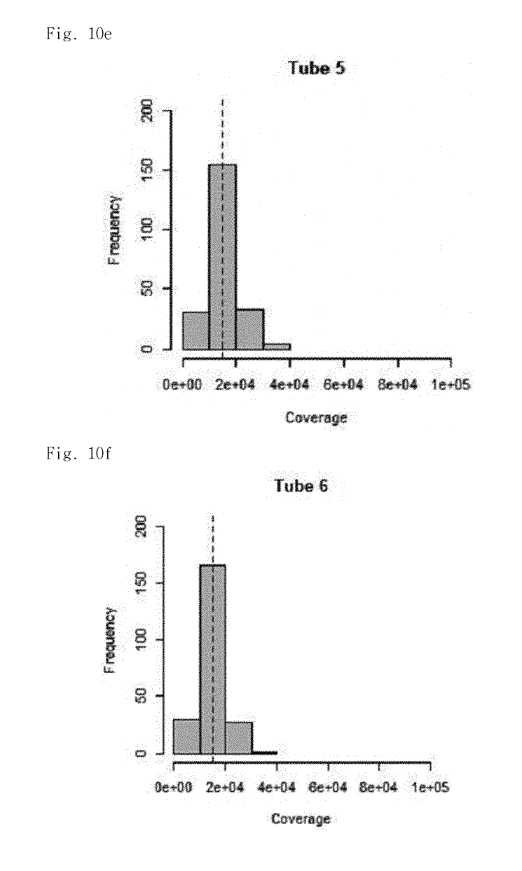

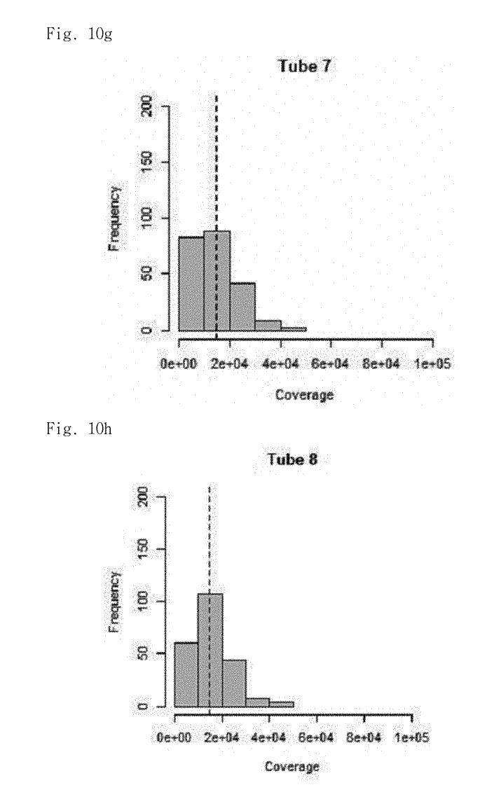

Beads collected in tubes for reading and reconstructing texts were amplified and sequenced on the Illumina HiSeq platform (FIGS. 10a, 10b, 10c, 10d, 10e, 10f, 10 g and 10 h). Low-quality raw reads were trimmed and expected oligonucleotide lengths were examined. In Illumina data, two reads were duplicated using 150 bp paired-end reads to filter out sequencing errors. After filtering out perfect index or address sequences (usually substitution errors), the consensus bases of data sequences were generated using the majority selection rule. It was confirmed that the original text was restored to 100% accuracy.

Compared with conventional microarray methods in which a large number of oligonucleotides need to be synthesized one by one to prepare an oligonucleotide pool, according to a method of the present invention, a single synthesis step is enough to prepare oligonucleotides having various combinations of base sequences. Therefore, hundreds to tens of thousands of synthesis steps can be reduced. In addition, since available base sequence combinations increase exponentially with increasing sequencing throughput, the method of the present invention can be widely used for effective DNA synthesis, preparation of large quantities of genetic materials, preparation of storage media using DNA, and the like.

What is claimed is:

1. A method of preparing an oligonucleotide pool comprising a plurality of clonal oligonucleotides, the method comprising:
synthesizing a plurality of clonal oligonucleotides from one oligonucleotide; and
performing next-generation sequencing on the synthesized clonal oligonucleotides to identify an entire base sequence of each of the clonal oligonucleotides,
wherein the synthesizing is performed so that the clonal oligonucleotide contains a random space,
wherein the random space has a length of R mer and consists of any one base sequence selected from the group consisting of 4^{R} base sequences that can be made up of a combination of A, T, C, and G.

2. The method according to claim 1, further comprising selecting a clonal oligonucleotide comprising a random space consisting of a desired base sequence among the clonal oligonucleotides, all base sequences of which have been identified.

3. The method according to claim 1, wherein the synthesizing is performed only once, and the oligonucleotide pool comprises 2 to 4^{R} clonal oligonucleotides having different base sequences in random spaces.

4. The method according to claim 1, wherein both termini of the one oligonucleotide comprise adaptor spaces in which adaptor sequences for next-generation sequencing (NGS) are present.

5. The method according to claim 1, wherein the one oligonucleotide has a total length of 100 mer or more.

6. The method according to claim 1, wherein the R is an integer of 2 to 20.

7. The method according to claim 1, wherein the one oligonucleotide consists of a base sequence corresponding to SEQ ID NO: 1 or a base sequence corresponding to SEQ ID NO: 2.

8. A method of storing information in DNA, comprising:
synthesizing a plurality of clonal oligonucleotides from one oligonucleotide;
performing next-generation sequencing on the synthesized clonal oligonucleotides to identify an entire base sequence of each of the clonal oligonucleotides;
performing mapping by inputting, on x-y coordinates, a base sequence of a random space of each of the clonal oligonucleotides, all base sequences of which have been identified; and
selecting, from a sequencing plate, a clonal oligonucleotide comprising a base sequence that matches a base sequence encoding information to be stored, wherein the synthesizing is performed so that the clonal oligonucleotide contains a random space, wherein
the random space has a length of R bp and consists of any one base sequence selected from the group consisting of 4^{R} base sequences that can be made up of a combination of A, T, C, and G, and
the base sequence of the random space consists of an address sequence encoding address information and a data sequence encoding data information.

9. The method according to claim 8, further comprising encoding information (target information) to be stored in DNA as a base sequence made up of A, T, C, and G.

10. An oligonucleotide capable of generating an oligonucleotide pool comprising a plurality of clonal oligonucleotides, wherein the oligonucleotide is one oligonucleotide consisting of a base sequence corresponding to SEQ ID NO: 1 or a base sequence corresponding to SEQ ID NO: 2.
